(19) United States
(12) Patent Application Publication
(10) Pub. No.: US 2025/0177975 A1
(43) Pub. Date: Jun. 5, 2025

(54) MULTIPLEX CARTRIDGE FOR DETECTION OF VIRAL AND/OR BACTERIAL NUCLEIC ACIDS AND HUMAN OR ANIMAL SINGLE NUCLEOTIDE POLYMORPHISMS

(71) Applicant: DNANUDGE LIMITED, London (GB)

(72) Inventors: Christofer TOUMAZOU, London (GB); Rashmita SAHOO, London (GB)

(21) Appl. No.: 18/843,003

(22) PCT Filed: Mar. 1, 2023
(86) PCT No.: PCT/EP2023/055227

§ 371 Date: Aug. 30, 2024

Related U.S. Application Data
(63) Continuation of application No. 17/684,005, filed on Mar. 1, 2022, now abandoned.

(51) Int. Cl. B01L 3/00; B01L 7/00; C12Q 1/686; C12Q 1/70

(52) U.S. Cl. CPC B01L 3/502715; B01L 7/52; C12Q 1/70; B01L 2200/16; B01L 2300/0803; C12Q 1/686

(57) ABSTRACT

A disposable cartridge comprising a sample receiving chamber for receiving a human or animal sample and an analysis unit (AU) having a multiplicity of reaction sites for providing a detectable indication of the presence in said sample of (a) one or more viral or bacterial nucleic acid sequences; and (b) one or more defined single nucleotide polymorphisms in the human or animal genome.

### FIELD OF THE INVENTION

The invention relates to a disposable cartridge that is capable of detecting both viral nucleic acid and at least one single nucleotide polymorphism (SNP) associated with disease or a disease risk from a human or animal in a sample. The disposable cartridge may additionally be capable of detecting bacterial nucleic acids, either in addition or alternative to viral nucleic acids. This cartridge has utility in providing diagnostic information by identifying samples infected with one or more viruses and/or bacteria; and can also simultaneously provide prognostic or diagnostic information with regard to the disease associated with the SNP. The disease or disease risk associated with the SNP may further be linked with a poorer clinical outcome as a result of infection with the virus or bacteria. The disposable cartridge may also inform a medical practitioner on suitable courses of treatment based on the SNPs identified.

### BACKGROUND OF THE INVENTION

Molecular testing such as Reverse Transcriptase PCR (RT-PCR) remains the standard of care for detection of viruses, such as SARS-CoV-2, due to its higher sensitivity and specificity [1]. However, standard laboratory RT-PCR can be time consuming and requires samples to be processed in centralised laboratory facilities. Factoring in sample transportation time and the requirement to process samples in batches means that the turnaround time for laboratory RT-PCR testing can often exceed 24 hours. Point-of-care diagnostic tests, which can be run outside of traditional laboratory settings have the potential to accelerate clinical decision making and enable effective triage and infection control measures in frontline clinical and community settings.

Co-morbidities, that is the concurrence of more than one disease or disorder in the same individual, can have a significant effect on the clinical outcome of a patient infected with a bacterial or viral infection. For example, there are a number of co-morbidities that are clinical risk factors indicative of a poorer clinical outcome in SARS-CoV2 infections. For example, it has been widely reported that SARS-CoV-2 patients who are overweight or obese have a greater risk of severe infection, and poorer clinical outcome, as compared to patients who are not overweight or obese. Body mass index (BMI) can therefore be used as an indicator of prognosis for SARS-CoV-2 patients.

Whilst mortality may be higher in overweight or obese patients, patients whose BMI is considered ‘normal’ may nevertheless suffer severe symptoms from SARS-CoV-2 infection, and may experience ongoing symptoms after recovery (known as ‘long COVID’) or even death. There is therefore a need to identify patients who may be medically predisposed towards severe infection. This may be additionally useful so medical practitioners can adapt their treatment plans accordingly at the earliest available opportunity. The association between, for example, obesity and poorer clinical outcome for, for example, SARS-CoV-2 patients also highlights the importance of reducing the prevalence of obesity and other chronic conditions.

Beyond SARS-CoV-2, obesity is a chronic condition that has many health implications and is associated with the development of detrimental diseases including diabetes and cardiovascular diseases. Preceded only by smoking, obesity is also the second biggest cause of certain types of cancer. Causes of obesity are complex. Both modifiable lifestyle factors, including diet and physical activity, alongside constant factors, such as genetics, influence a person's likelihood of developing obesity. The FTO gene, also known as the ‘Obesity gene’, is one of several well-established genetic contributors with common variants that affect obesity susceptibility.

Molecular testing such as PCR remains the standard for genotyping, for instance by means of detection of single nucleotide polymorphisms that are associated with a disease or disease risk. However, similar to RT-PCR, standard laboratory PCR can be time consuming and requires samples to be processed in centralised laboratory facilities. Factoring in sample transportation time and the requirement to process samples in batches means that the turnaround time for laboratory PCR testing can often exceed 24 hours. Moreover, genotyping is not a current standard of care when performing a diagnosis. However, it is recognised that genetic analysis of patients can inform a patient's treatment plan (i.e. personalised medicine), which can ultimately improve the prognosis for a patient. Genetic analysis of an individual can also be used to guide life-style choices to prevent or again improve the prognosis for an individual. There is therefore a need to improve the availability of diagnostic and prognostic medical devices.

Finally, there lies a particular problem where there is a need to simultaneously detect both RNA and DNA in a sample—for example, an RNA virus and a SNP from a patient sample. Typically, to detect both RNA and DNA, RT-PCR is required for the former and PCR for the latter. In the laboratory setting, RT-PCR and PCR are performed separately. The practitioner generally either isolates RNA or DNA from a biological sample, before going on to amplify nucleic acids. However, for a point-of-care diagnostic for use as above, there is a need to provide a device capable of performing both RNA and DNA nucleic acid analysis simultaneously. Performing PCR and RT-PCR in a single process (i.e. one-pot PCR and RT-PCR) leads to various challenges in accurately replicating the RNA and DNA respectively comprises in the sample. Amplicons arising from incomplete extension, along with off-target annealing of primers/incomplete amplicons to target nucleic acid molecules can lead to the generation of unwanted nucleic acid sequences. For instance, primers intended for RT-PCR may interfere with primers for PCR and/or amplicons generated during the PCR reaction. These issues are particularly apparent where RT-PCR primers and/or PCR primers are used in excess. Where RT-PCR and PCR are performed in a single process (i.e. on-pot) one or more of these issues may be a problem. A single process which is suitable for RT-PCR and PCR to amplify RNA and DNA (and/or cDNA) templates respectively is required.

The present invention obviates or alleviates one or more of the above-mentioned problems.

### SUMMARY OF INVENTION

According to a first aspect of the present invention, there is provided a disposable cartridge comprising a sample receiving chamber for receiving a human or animal sample; one or more further chambers containing reagents and fluids; and an analysis unit (AU) having a multiplicity of reaction sites; the cartridge being operable to move fluids between the chambers and the AU and thereby generate at said reaction sites a detectable indication of the presence in said sample of (a) one or more viral or bacterial nucleic acid sequences; and (b) one or more defined single nucleotide polymorphisms in the human or animal genome; and wherein the one or more SNPs are indicative of a disease or a disease risk or other physiological condition in the human or animal, and wherein the disease or physiological condition is indicative of a poorer clinical outcome associated with a disease or other condition caused in the human or animal by the virus or bacteria.

The present invention provides a quick and efficient ‘sample-to-answer’ diagnosis of a bacterial or viral infection, combined with analysis of single nucleotide polymorphisms (SNPs) associated with a disease or disease risk. The present invention thus provides a diagnosis, whilst simultaneously providing an indication of disease or disease risk and an indication of clinical outcome with regard to recovery from said bacterial or viral infection. The SNP analysis may also reveal a medical predisposition, which rules out certain courses of treatment for the viral or bacterial infection and/or which means routine courses of treatment for the viral or bacterial infection need to be personalised according to the patient's DNA. Moreover, the SNP analysis may reveal an indication of likelihood of developing serious disease in the future and/or the future risk of suffering seriously from particular viral or bacterial infections. Such analysis can be used to guide life-style choices to mitigate such risks. The present invention thus provides a test that can be performed outside of traditional laboratory settings, avoids the need for any laboratory facilities or sample preparation, can be used at the point-of-care with a patient, and provides quick results in real-time.

The disposable cartridge described herein is particularly suited for point-of-care tests, which may be conducted in hospitals, surgeries, pharmacies, shops, schools, workplaces etc. In embodiments the cartridge is a single-use disposable cartridge.

In embodiments, the cartridge is configured to perform single nucleotide polymorphism genotyping; and to detect one or more viral or bacterial nucleic acid sequences. The cartridge may be configured to detect one or more viral nucleic acid sequences, wherein the viral nucleic acid is RNA. The cartridge may be configured to generate complementary DNA (cDNA) from the one or more viral RNA sequences. In embodiments, the cartridge is configured to generate cDNA from one or more viral nucleic acid sequences at a temperature of from 40 to 60° C., optionally 45 to 55° C., and optionally still about 50° C., for a duration of 2 to 10 minutes, optionally 3 to 7 minutes, optionally still about 5 minutes. Optionally, the cartridge is configured to generate cDNA from one or more viral nucleic acid sequences at a temperature of about 50° C. for about 5 minutes.

In embodiments, the cartridge is configured to generate complementary DNA (cDNA) from one or more viral RNA sequences; and then to perform DNA amplification of said cDNA concurrently with DNA amplification of said one or more defined single nucleotide polymorphisms in the DNA of the human or animal sample. The cartridge may be configured to heat the cDNA after generation of cDNA but before DNA amplification to a temperature of from 80 to 110° C., optionally from 90 to 100° C., and optionally still about 95° C., for a duration of 10 seconds to 120 seconds, optionally about 20 seconds.

The cartridge may be configured to perform DNA amplification for from 10 to 100 cycles, optionally from 20 to 80 cycles, optionally from 30 to 60 cycles (e.g. about 40 cycles). Each cycle may comprise a denaturation step having a duration of from 1 10 second to 120 seconds (e.g. from 2 to 5 seconds), and an annealing step having a duration from 30 seconds to 120 seconds (e.g. about 60 seconds). In embodiments, the cartridge is configured to perform DNA amplification for 40 cycles, each cycle comprising a denaturation step having a duration of about 2 seconds, and an annealing step having a duration of about 60 seconds.

In embodiments, the disease or other condition caused by the virus or bacteria infection is a respiratory disease. The respiratory disease may be a viral infection and the virus is a common cold, influenza, respiratory syncytial virus, adenovirus, or coronavirus. The virus may be a coronavirus, preferably SARS, SARS-CoV-2 or MERS. In embodiments, the coronavirus is SARS-CoV-2.

Any number of reaction sites may be provided in the analysis unit, depending on the desired number of viral or bacterial nucleic acids sequences to be detected, the number of SNPs desired to be detected, and the desired number of replicates thereof. In embodiments, from 50 to 150 reaction sites are provided in the analysis unit, optionally 60 to 100 (e.g. around 70, around 90).

In embodiments, the analysis unit's reaction sites may resemble a well-plate (e.g. a 48-well plate, a 96 well-plate), wherein each well is a blank or a reaction site.

In embodiments, at least one reaction site comprises at least one nucleic acid probe sequence capable of detecting at least one viral or bacterial nucleic acid sequence; and at least one reaction site comprises at least one SNP nucleic acid probe sequence, capable of detecting the one or more SNPs in the human or animal genome. In embodiments, at least two reaction sites comprise at least one nucleic acid probe sequence capable of detecting at least one viral or bacterial nucleic acid sequence, optionally at least three reaction sites, and optionally still at least four reaction sites (e.g. 5 reaction sites, 6 reaction sites, 7 reaction sites). In embodiments, at least two reaction sites comprises at least one SNP nucleic acid probe sequence, capable of detecting the one or more SNPs in the human or animal genome, optionally at least three reaction sites, and optionally still at least four reaction sites (e.g. 5 reaction sites, 6 reaction sites, 7 reaction sites).

In embodiments, at least one reaction site comprises a first nucleic acid probe sequence capable of detecting at least one viral or bacterial nucleic acid sequence; and at least one reaction site comprises a second nucleic acid probe sequence capable of detecting at least one viral or bacterial nucleic acid sequence. The first nucleic acid probe and second nucleic acid probe may each independently target the same viral or bacterial nucleic acid sequence, or a different viral or bacterial nucleic acid sequence. One or more further nucleic acid probes (e.g. a third, fourth, fifth, sixth nucleic acid probes) capable of detecting at least one viral or bacterial nucleic acid sequence may also be provided in one or more further reaction sites. Similarly, the one or more further nucleic acid probes may each independently target the same viral or bacterial nucleic acid sequence, or a different viral or bacterial nucleic acid sequence.

In embodiments, at least one reaction site comprises a first SNP nucleic acid probe sequence, capable of detecting the one or more SNPs in the human or animal genome; and at least one reaction site comprises a second SNP nucleic acid probe sequence, capable of detecting the one or more SNPs in the human or animal genome. The first SNP nucleic acid probe and second SNP nucleic acid probe may each independently target the SNP, or a different SNP. One or more further SNP nucleic acid probes (e.g. a third, fourth, fifth, sixth nucleic acid probes) capable of detecting one or more SNPs may also be provided in one or more further reaction sites. Similarly, the one or more further nucleic acid probes may each independently target the SNP, or a different SNP.

In embodiments, at least one of the reaction sites comprising at least one SNP nucleic acid probe sequence is used as a control for DNA amplification. In alternative embodiments, at least one reaction site may comprise at least one nucleic acid probe sequence capable of detecting at least one control nucleic acid sequence.

In embodiments, the at least one reaction sites comprise at least one or more primer nucleic acid sequences. The primer nucleic acid sequences may be capable of targeting at least a fragment of the one or more viral or bacterial nucleic acid sequences to facilitate PCR amplification. The primer nucleic acid sequences may be capable of targeting at least a fragment of the one or more viral or bacterial nucleic acid sequences to facilitate PCR amplification. The primer nucleic acid sequences may be capable of targeting at least a fragment of the nucleic acid sequence associated with one or more defined single nucleotide polymorphisms to facilitate PCR amplification. The at least one or more primer nucleic acid sequences may comprise a forward primer nucleic acid sequence and a reverse primer nucleic acid sequence. In each reaction site, the at least one or more primer nucleic acid sequences may target a fragment of the nucleic acid sequence (i.e. viral or bacterial or SNP), wherein the at least one nucleic acid probe sequence also targets said nucleic acid sequence (viral or bacterial or SNP nucleic acid sequence) or a fragment thereof.

In embodiments, at least one of the nucleic acid probe sequences and/or at least one of the primer nucleic acid sequences is labelled with a fluorescent marker.

In embodiments, the least one nucleic acid probe sequence capable of detecting at least one viral or bacterial nucleic acid is a probe sequence capable of detecting at least one viral nucleic acid. In embodiments, the at least one viral nucleic acid comprises at least a fragment of a nucleic acid sequence of SARS-CoV-2.

In embodiments, at least one reaction site comprises at least one SNP nucleic acid probe sequence, capable of detecting the one or more SNPs in the human or animal genome, wherein the at least one SNP nucleic acid probe sequence is capable of detecting one or more SNPs associated with fat mass and obesity-associated (FTO) gene sequences, or specific fragments thereof. The at least one SNP nucleic acid probe sequence may be capable of binding to at least one nucleic acid sequence selected from rs9937053 (A/G), rs9939973 (A/G), rs9940128 (A/G), rs1421085 (C/T), rs1558902 (A/T), rs1121980 (A/G), rs7193144 (C/T), rs8043757 (T/A), rs8050136 (A/C), rs3751812 (T/G), rs9923233 (C/G), rs9926289 (A/G), rs9939609 (A/T), rs7185735 (G/A), rs9931494 (G/C), rs17817964 (T/C), rs9930506 (G/A), rs9932754 (C/T), rs9922619 (T/G), rs7204606 (C/T) and rs12149832 (A/G) alleles. In embodiments, the at least one SNP nucleic acid probe sequence is capable of binding to nucleic acid sequence rs1558902 (A/T).

In embodiments, the one or more single nucleotide polymorphisms (SNPs) in the human or animal genome to be detected comprises at least 2, 3, 4, 5, 6, 7, or 8 specific nucleic acid sequences which are specific to the disease or other physiological condition in the human or animal. In embodiments, the one or more SNPs to be detected comprises at most 4, 6, 8, 10, or 12 specific nucleic acid sequences which are specific to the disease or other physiological condition in the human or animal.

In embodiments, the disease or other physiological condition in the human or animal is one or more of obesity, diabetes, cardiovascular disease, cerebrovascular disease, respiratory disease, kidney disease and malignancy. The disease or other physiological condition in the human or animal may be obesity, and the one or more SNPs may be, or may include, fat mass and obesity-associated (FTO) gene sequences, or specific fragments thereof.

In embodiments, the disease or other physiological condition in the human or animal is obesity, the one or more SNPs is fat mass and obesity-associated (FTO) gene sequences or specific fragments thereof, and the one or more viral or bacterial nucleic acid sequences is a viral nucleic acid sequence of SARS-CoV-2.

In embodiments, the analysis unit may comprise at least two (e.g, 3, 4, 5, 6, 7, 8, 9) reaction sites comprising a first nucleic acid probe sequence capable of detecting a nucleic acid sequence of SARS-CoV-2; and at least two (e.g, 3, 4, 5, 6, 7, 8, 9) reaction sites comprising a second nucleic acid probe sequence capable of detecting a nucleic acid sequence of SARS-CoV-2. In embodiments, at least two (e.g, 3, 4, 5, 6, 7, 8, 9) reaction sites comprising a third nucleic acid probe sequence capable of detecting a nucleic acid sequence of SARS-CoV-2 are provided. In embodiments, at least two (e.g, 3, 4, 5, 6, 7, 8, 9) reaction sites comprising a fourth nucleic acid probe sequence capable of detecting a nucleic acid sequence of SARS-CoV-2 are provided. In embodiments, at least two (e.g, 3, 4, 5, 6, 7, 8, 9) reaction sites comprising a fifth nucleic acid probe sequence capable of detecting a nucleic acid sequence of SARS-CoV-2 are provided. In embodiments, at least two (e.g, 3, 4, 5, 6, 7, 8, 9) reaction sites comprising a sixth nucleic acid probe sequence capable of detecting a nucleic acid sequence of SARS-CoV-2 are provided. Each of the first to sixth nucleic acid probe sequences may target different regions of a nucleic acid sequence of SARS-CoV-2.

The analysis unit may further comprise at least two (e.g, 3, 4, 5, 6, 7, 8, 9) reaction sites comprising a first SNP nucleic acid probe sequence capable of detecting one or more SNPs associated with fat mass and obesity-associated (FTO) gene sequences, or specific fragments thereof; and at least two (e.g, 3, 4, 5, 6, 7, 8, 9) reaction sites comprising a second SNP nucleic acid probe sequence capable of detecting one or more SNPs associated with fat mass and obesity-associated (FTO) gene sequences. The first and second SNP nucleic acid probe sequences may be capable of binding to at least one nucleic acid sequence selected from from rs9937053 (A/G), rs9939973 (A/G), rs9940128 (A/G), rs1421085 (C/T), rs1558902 (A/T), rs1121980 (A/G), rs7193144 (C/T), rs8043757 (T/A), rs8050136 (A/C), rs3751812 (T/G), rs9923233 (C/G), rs9926289 (A/G), rs9939609 (A/T), rs7185735 (G/A), rs9931494 (G/C), rs17817964 (T/C), rs9930506 (G/A), rs9932754 (C/T), rs9922619 (T/G), rs7204606 (C/T) and rs12149832 (A/G) alleles. One or more further SNP nucleic acid probe sequences (e.g. third, fourth, fifth, sixth) may be provided in one or more further wells.

Advantageously, using multiple nucleic acid probe sequences for detecting one or more viral or bacterial nucleic acid sequences, and/or using multiple nucleic acid probe sequences for detecting one or more defined single nucleotide polymorphisms in the human or animal genone, increases the accuracy of the calling results, as described in more detail below.

In embodiments, the sample is nasopharyngeal or sputum or saliva. In embodiments, the sample receiving chamber is configured to receive a sample head of a swab. The swab may be a buccal, nasopharyngeal, or oropharyngeal swab.

In embodiments, the cartridge is configured to be introduced into a processing unit, optionally wherein the processing unit is a NudgeBox™ analyser.

In embodiments, the one or more further chambers comprise at least a chamber comprising a lysis buffer, a chamber containing a wash buffer, a chamber containing an elution buffer, and a chamber comprising a lyophilised composition comprising reagents for RT-PCR and PCR.

The lysis buffer may comprise at least one chaotropic agent, wherein the concentration of the chaotropic agent in the lysis buffer is from 2 M to 6 M; at least one acetate salt, wherein the concentration of the acetate salt in the lysis buffer is from 0.1 to 2 mM; at least one chelating agent, wherein the concentration of the chelating agent in the lysis buffer is from 5 mM to 50 mM; and at least one surfactant, wherein the concentration of the surfactant in the lysis buffer is from 0.1 to 2 mM. The lysis buffer may be an aqueous lysis buffer, optionally wherein the lysis buffer has a pH of from 5.0 to 7.0, preferably 5.5 to 6.5, and preferably still about 6.0. In embodiments, the concentration of the chaotropic agent is from 3 to 5 M, preferably about 4 M. In embodiments, the chaotropic agent is guanidine hydrochloride. In embodiments, the concentration of the acetate salt is from 10 mM to 40 mM, optionally from 20 mM to 30 mM, preferably about 25 mM. In embodiments, the acetate salt is sodium acetate. In embodiments, the concentration of the chelating agent is from 0.5 to 1.5 mM, preferably about 1 mM. In embodiments, the chelating agent is EDTA. In embodiments, the concentration of the surfactant is from 1 to 2 mM, preferably about 1.5 mM. In embodiments, the surfactant is a non-ionic surfactant, optionally wherein the non-ionic surfactant is Triton X-100. In embodiments, the lysis buffer is an aqueous lysis buffer having a pH around 6.0, and comprises about 4 M guanidine hydrochloride; about 25 mM sodium acetate salt; about 1 mM EDTA; and about 1.5 mM non-ionic surfactant.

In embodiments, the wash buffer comprises from 0.1 to 20 mM tris(hydroxymethyl)aminomethane and from 50 to 90 wt % of a C₁-C₃ alcohol, preferably from 60 to 80 wt % of a C₁-C₃ alcohol. In embodiments, the concentration of tris(hydroxymethyl)aminomethane in the wash buffer is from 1-15 mM, optionally from 5-15 mM, preferably about 10 mM. In embodiments, the C₁-C₃ alcohol is ethanol. In embodiments, the wash buffer is an aqueous wash buffer, and wherein the wash buffer has a pH of from 6.0-9.0, preferably 7.0-8.0, and preferably still about 7.5. In embodiments, the wash buffer is an aqueous wash buffer having a pH around 7.5, and comprises 10 mM tris(hydroxymethyl)aminomethane and from 60 to 80 wt % ethanol.

In embodiments, the elution buffer comprises from 0.1 to 20 mM tris(hydroxymethyl)aminomethane; at least one chelating agent, wherein the concentration of the chelating agent in the lysis buffer is from 0.01 mM to 1 mM; and at least one surfactant, wherein the concentration of the surfactant in the lysis buffer is from 0.1 to 2 mM. In embodiments, the concentration of tris(hydroxymethyl)aminomethane in the elution buffer is from 1-15 mM, optionally from 5-15 mM, preferably about 10 mM. In embodiments, the concentration of the chelating agent in the elution buffer is from 0.05 mM to 0.3 mM, preferably about 0.1 mM. In embodiments, the chelating agent is EDTA. In embodiments, the concentration of the surfactant in the elution buffer is from 1 to 2 mM, preferably about 1.5 mM. In embodiments, the surfactant is a non-ionic surfactant, optionally wherein the non-ionic surfactant is Triton X-100. In embodiments, the elution buffer is an aqueous elution buffer, and wherein the elution buffer has a pH of from 6.0 to 10.0, preferably from 7.0 to 9.0, and preferably still about 8.0. In embodiments, the elution buffer is an aqueous elution buffer having a pH around 8.0, and comprises about 10 mM tris(hydroxymethyl)aminomethane; about 0.1 mM EDTA; and about 1.5 mM non-ionic surfactant.

In embodiments, the lyophilised composition comprises deoxynucleoside triphosphates (dNTPs i.e. a mixture of dATP, dCTP, dGTP and dTTP), DNA polymerase and Reverse transcriptase. The lyophilised composition may comprise one or more salts selected from buffer salts (e.g. Tris), one or more sulfate salts (e.g. MgSO₄), one or more chloride salts (e.g. NaCl, KCl). The lyophilised composition may comprise bovine serum albumin (BSA). The lyophilised composition may comprise an RNA-ase inhibitor (e.g. an inhibitor of Ribonuclease P (RNaseP)). dNTPs may optionally be labelled with a fluorescent marker.

According to a second aspect of the present invention, there is provided a method for detecting the presence of (a) one or more viral or bacterial nucleic acid sequences; and (b) one or more defined single nucleotide polymorphisms (SNPs) in the human or animal genome, in a human or animal sample, the method comprising obtaining the human or animal sample; inserting the sample into the sample receiving chamber of a disposable cartridge according to the first aspect of the present invention, and sealing the sample within the sample receiving chamber; inserting the cartridge into a processing unit, optionally wherein the processing unit is a NudgeBox™ analyser; extracting RNA and DNA from the human or animal sample; and performing RT-PCR and PCR reactions on the sample within the disposable cartridge; wherein the one or more SNPs are indicative of a disease or a disease risk or other physiological condition in the human or animal, and wherein the disease or physiological condition is indicative of a poorer clinical outcome associated with a disease or other condition caused in the human or animal by the virus or bacteria; and wherein the processing unit is programmed to concurrently perform RT-PCR and PCR reactions on the sample within the disposable cartridge; and to analyse the detectable indication.

In embodiments, the method comprises contacting the sample with a lysis buffer to provide a lysate comprising RNA and DNA, wherein the lysis buffer is an aqueous lysis buffer having a pH around 6.0, and comprising about 4 M guanidine hydrochloride; about 25 mM sodium acetate salt; about 1 mM EDTA; and about 1.5 mM non-ionic surfactant. In embodiments, the method comprises contacting the lysate with a solid support to immobilise the DNA and RNA. In embodiments, the method comprises washing the solid support with a wash buffer, wherein the wash buffer is an aqueous wash buffer having pH a around 7.5 comprising 10 mM tris(hydroxymethyl)aminomethane and from 60 to 80 wt % ethanol. In embodiments, the method comprises contacting the solid support with an elution buffer to remove the DNA and RNA from the solid support and to provide an eluent comprising the DNA and RNA, wherein the elution buffer is an aqueous elution buffer having a pH around 8.0, and comprises about 10 mM tris(hydroxymethyl)aminomethane; about 0.1 mM EDTA; and about 1.5 mM non-ionic surfactant. In embodiments, the method comprises contacting the eluent with a lyophilised mixture comprising dNTPs, reverse transcriptase and DNA polymerase to provide a reconstituted mixture. In embodiments, the method comprises heating the reconstituted mixture to 50° C. for 5 minutes to generate cDNA to provide a cDNA/DNA mixture. In embodiments, the method comprises inactivating the reverse transcriptase enzyme and activating the DNA polymerase by heating the cDNA/DNA mixture to 95° C. for 20 seconds to provide a cDNA/DNA/DNA polymerase mixture. In embodiments, the method comprises contacting the cDNA/DNA/DNA polymerase mixture with a primer and probe composition comprising at least one primer nucleic acid sequence and at least one nucleic acid probe sequence capable of detecting at least one viral or bacterial nucleic acid sequence, optionally wherein the probe sequence is labelled with a fluorescent marker; and at least one SNP nucleic acid probe sequence, capable of detecting the one or more SNPs in the human or animal genome, optionally wherein the probe sequence is labelled with a fluorescent marker; and a plurality of DNA nucleotides. In embodiments, the method comprises performing DNA amplification for 40 cycles, each cycle comprising a denaturation step for 2 seconds at 95° C. and an annealing step for 30 seconds at 60° C. to provide an amplified DNA mixture. In embodiments, the method comprises analysing the amplified DNA mixture to detect the presence of the one or more viral or bacterial nucleic acid sequences; and (b) the one or more defined single nucleotide polymorphisms (SNPs) in the human or animal genome in the human or animal sample.

According to the third aspect of the present invention, there is provided a system for detecting the presence of (a) one or more viral or bacterial nucleic acid sequences; and (b) one or more defined single nucleotide polymorphisms (SNPs) in the human or animal genome, in a human or animal sample, the system comprising a disposable cartridge according to the first aspect of the present invention; and a processing unit, wherein the processing unit is configured to effect extraction of nucleic acids from the human or animal sample inside the disposable cartridge, effect RT-PCR and PCR on said nucleic acids inside the disposable cartridge, and to detect formation of amplicons arising from PCR. In embodiments, the processing unit detects amplification by means of a camera. In embodiments, the processing unit comprises software configured to detect formation of amplicons in the PCR reactions. In embodiments, the amplicons are fluorescent, and the processing unit comprises a camera capable of detecting fluorescence.

Further embodiments of the present teaching are defined by the following detailed description of exemplary embodiments, which should not be construed as limiting.

### BRIEF DESCRIPTION OF THE FIGURES

The invention is further described in the following non-limiting figures:

FIG. 1 depicts a disposable cartridge (left), and the same disposable cartridge with an Isohelix swab (with stopper and bung) inserted into the sample chamber (right);

FIG. 2 depicts a cross-sectional planview of a disposable cartridge according to the present invention comprising the analysis unit and sample preparation unit.

FIG. 3 is a partial cross-sectional plan view of a sample preparation unit comprised in the disposable cartridge according to the invention;

FIG. 4 depicts plan view of an analysis unit comprised in the disposable cartridge according to the invention;

FIG. 5 is an exemplary arrangement of reaction sites in the analysis unit;

FIG. 6 depicts a NudgeBox (28×15·5×13·5 cm; 5 kg) and a disposable cartridge (Nucleic Acid (RNA/DNA) Cartridge) (25×78×85 mm; 40 g);

FIG. 7: Number of replicates amplifying on a DnaNudge platform not according to the present invention but provided for comparative purposes (i.e. NudgeBox and a disposable cartridge not according to the invention) for nasopharyngeal and sputum samples. The data does not suggest that nasopharyngeal or sputum samples present a consistently higher viral load, with significant patient-to-patient variation. The average number of replicates across all samples was similar for both sputum (mean replicates=24.7) and nasopharyngeal (mean replicates=27.1), with the number of replicates amplifying ranging from 3-54 for sputum samples and 3-56 for nasopharyngeal samples.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The disposable cartridges according to the invention provide an all-in-one solution for detecting viral nucleic acid and at least one human SNP associated with a disease or disease risk simultaneously, using one single disposable cartridge. Optionally or alternatively, the disposable cartridges may also be configured to detect bacterial DNA. The cartridge is equipped with reagents which enable preparation of the biological sample under optimised conditions such that both viral nucleic acid and patient SNP analysis are able to be performed concurrently. Typically the viral nucleic acid may be RNA, such that in essence, the disposable cartridges of the present invention are optimised for subsequent thermal cycling of RT-PCR and PCR reactions, in a single reaction process (i.e. one-pot RT-PCR and PCR).

The viral nucleic acid may be RNA or DNA. In one embodiment, the virus is an RNA virus. The virus may be a double-stranded RNA virus, a positive-sense single stranded RNA virus or a negative-sense single stranded RNA virus. Examples of RNA viruses include severe acute respiratory syndrome coronavirus (SARS-CoV), Middle East respiratory syndrome-related coronavirus (MERS-CoV), severe acute respiratory syndrome coronavirus 2 (SARS-CoV-2), Dengue virus, hepatitis C virus (HCV), hepatitis E virus (HEV), West Nile virus, Ebola virus, lyssaviruses, poliovirus, mumps virus and measles virus.

The SNP may be associated with a disease. That is, the presence of the genetic variant—i.e. the SNP—is indicative of the disease. In this example, the SNP may be a causal variant. Alternatively, the SNP may be associated with a disease risk. That is, the presence of the genetic variant is indicative of a higher risk or higher overall risk of developing a disease compared to the risk in an individual without this genetic variant.

The SNP may be associated with any disease or disease risk. However, in one embodiment, the SNP is additionally associated with a poorer clinical outcome as a result of the bacterial or viral infection. Such SNPs would be well known to the skilled person, and therefore methods for the identification of suitable SNPs does not need to be further defined herein.

In one example, where the viral infection is a Coronavirus, and in particular, SARS-CoV-2, the SNP may be associated with one of the following conditions or risk of developing one of the following conditions: obesity, hypertension, diabetes, cardiovascular disease, cerebrovascular disease, respiratory disease, kidney disease and malignancy.

In one example, the at least one SNP may be associated with obesity or risk of developing obesity. Multiple genetic variations in intron 1 of the fat mass and obesity genes (FTO genes) have been linked with severe obesity, and in particular increased BMI, body rate fat, waist circumference, hip circumference and energy intake. In one embodiment, the at least one SNP may be selected from the following SNPs in the FTO gene: rs9937053 (A/G), rs9939973 (A/G), rs9940128 (A/G), rs1421085 (C/T), rs1558902 (A/T), rs1121980 (A/G), rs7193144 (C/T), rs8043757 (T/A), rs8050136 (A/C), rs3751812 (T/G), rs9923233 (C/G), rs9926289 (A/G), rs9939609 (A/T), rs7185735 (G/A), rs9931494 (G/C), rs17817964 (T/C), rs9930506 (G/A), rs9932754 (C/T), rs9922619 (T/G), rs7204606 (C/T) and rs12149832 (A/G) alleles. Identification of the presence of one or more of these alleles with the cartridge of the invention, can be used to determine if an individual is at a higher or lower genetic risk of developing obesity.

In one example, the FTO SNP is the rs1558902 (A/T) allele. The rs1558902 allele is closely associated with increased BMI, and to obesity. This single nucleotide variation is at a specific location on the genome (chr16: 53769662) and at that specific location nucleotide T or nucleotide A (allele T and allele A respectively) is present. The T allele is the common allele (wild type) and the A allele is the alternative one associated with increased BMI and obesity. Each individual has two sets of chromosomes (a paternal and a maternal), and so will also have two alleles with respect to this FTO SNP. A person with two Ts (TT) is a homogygote for the common allele, a person with one T and one A (TA) is a heterozygote, and a person with two As is a homozygote for the alternative allele. People that carry at least one copy of the A allele (ie TA, and AA) have increased genetic risk for obesity.

Although the description is primarily directed to the detection of SARS-CoV-2 and a FTO SNP (rs1558902) should not be construed as limiting. The teachings herein can easily be extended to the detection of any viral or bacterial infection that can be detected by way of one or more nucleic acid sequences which are associated with the infection. Similarly, the teachings can easily be extended to detection of a disease (or predisposition to a disease) that can be detected by way of one or more SNPs associated with said disease. The SNP may be indicative of the presence of disease, the severity of symptoms a patient is likely to suffer through the viral or bacterial infection, or of the patient's likelihood of suffering with a disease in future, which makes them susceptible to said viral or bacterial infection.

Examples of infections include respiratory infections caused by bacterial, viral or fungal agents. Examples of diseases include obesity, diabetes, hypertension, cardiovascular disease, cerebrovascular disease, respiratory disease, kidney disease and malignancy.

The disposable cartridges of the present invention may be structurally and mechanically configured in accordance with the disposable cartridges described in more detail in reference WO2018055407, and in Gilbani and Toumazou [2], to which the skilled reader is directed and which are incorporated herein by reference. The disposable cartridges according to the present invention may be operated as described in reference WO2018055407, and in Gilbani and Toumazou [2]. Moreover the disposable cartridges according to the present invention are configured to be used in conjunction with a processing unit (e.g. NudgeBox™), such as the processing unit described in reference WO2018055407, and in Gilbani and Toumazou [2]. The disposable cartridges of the present invention differ from what is disclosed in the prior art in the one or more reagents and fluids comprised in the one or more chambers, and in the reaction sites provided in the analysis unit. Moreover, the disposable cartridges of the present invention are provided with the necessary reagents, fluid and reaction sites, which enables combined RT-PCR and PCR tests.

Disposable cartridges suitable for use according to the present invention are described below by way of example only, with reference to the accompanying Figures. As mentioned above, the skilled person will appreciate that the structural and mechanical configuration of cartridges suitable for use in accordance with the present invention, along with the use and operation thereof, is described in detail in WO2018055407, which is incorporated herein by reference.

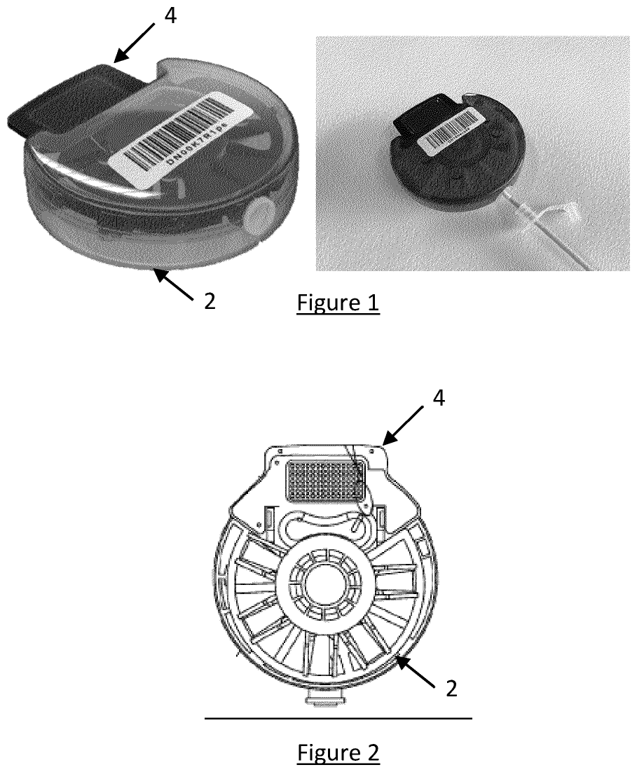

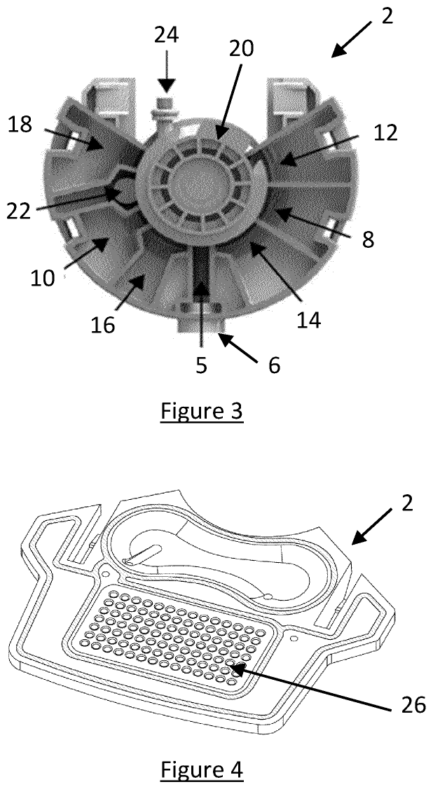

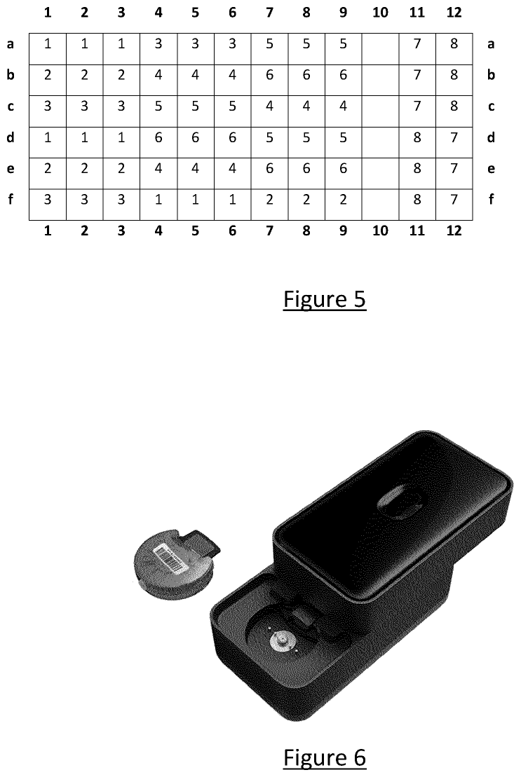

FIGS. 1 and 2 show disposable cartridges (1) according to the present invention. The disposable cartridges comprise a sample preparation unit (2) and an analysis unit (4). FIG. 3 shows a cross-section plan view of the sample preparation unit (2) (SPU). The sample preparation unit (SPU) receives the human or animal sample, extracts DNA and/or RNA therefrom, and purifies the DNA and/or RNA ready for PCR and RT-PCR respectively. The PCR and RT-PCR is performed in the analysis unit (AU) as illustrated in FIGS. 4 and 5 (discussed in more detail below).

The sample preparation unit (2) comprises a sample receiving chamber (5), which is accessible via an inlet (6). The inlet (6) and sample receiving chamber (5) are configured to receive the head of a swab (FIG. 1, right). The cartridge further comprises additional chambers containing reagents and fluids. With reference to FIG. 3, a chamber comprising a lysis buffer (8), a chamber comprising a wash buffer (10), and a chamber comprising an elution buffer (12) are provided. Those skilled in the art will appreciate that although these particular buffers are exemplified herein for the purposes of concurrent RNA/DNA extraction and purification, that alternative or additional reagents and fluids may be used. One or more further chambers (14, 16, 18) are also provided which may contain one or more additional or alternative reagents for the extraction and purification of RNA/DNA from the human or animal sample. Additionally or alternatively, one or more further chambers (14, 16, 18) may be provided to receive waste fluids and waste reagents as the sample preparation process progresses. The sample preparation unit (2) further comprises a rotating central member (20) comprising an interior (flow through) annular chamber (not illustrated; as described in WO2018055407). The interior annular chamber comprises a solid support (e.g. a silica frit) for binding RNA and/or DNA during the preparation process. Those skilled in the art will be aware of suitable solid supports for binding RNA and/or DNA in accordance with the present invention. For example, solid supports commercially available from SigmaAldruch (e.g. Whatman® glass microfiber filters, Grade 934-AH, WHA1827047) may be used.

The chambers comprising the lysis buffer (8), wash buffer (10) and elution buffer (12) can be selectively brought into fluid communication with the interior annular chamber, and optionally with one or more other chambers. Each chamber comprising a reagent or fluid (8, 10, 12) comprises a fluid outlet (not illustrated). The rotating central member (20) comprises at least one fluid inlet, which provides fluid access to the interior annular chamber. The body of the rotating central member (20) can occlude one or more of the fluid outlets of the chambers to prevent fluid communication between the fluid outlet of a chamber, and the fluid inlet of the rotating central member. By rotating the rotating central member, the fluid outlet of a chamber and the fluid inlet of the central member (20) can be selectively aligned to provide fluid communication between the chamber and the interior annular chamber of the central member. The central member (20) may comprise one or more additional inlets configured to selectively align with the fluid outlet of a first chamber and the fluid outlet of a second chamber, so as to provide concurrent fluid communication between at least two chambers and the interior annular chamber of the central member (20).

The chambers comprising the reagents and fluids therefore are configured to participate in various stages of a sample preparation procedure, such that separate fluids and reagents can be brought into contact with the sample in isolated reaction steps. Those skilled in the art will appreciate that several fluids or reagents may be desired for a single reaction step. Additionally or alternatively, a single reaction step may be performed in one or more chambers, the rotating central chamber, or a combination thereof.

Typically, each disposable cartridge comprises from 400 to 500 μL lysis buffer, from 400 to 500 μL wash buffer, and from 0.5 to 1.5 mL elution buffer. Typically, each cartridge contains 450 μL lysis buffer, 450 μL wash buffer, and 1 mL elution buffer.

The various fluids, reagents, lysate and eluent provided during the reaction steps may be directed between chambers and the annular chamber of the rotating central member (20) by means of generating pressure differentials (e.g. by means of a plunger arranged in a bore of the rotating central member, or by pneumatic means (e.g. a pump) and a plurality of communicating channels, as described in WO2018055407.

In use, as shown in FIG. 1, the user inserts the sample into the sample receiving chamber, which may be by means of a swab. The user can cut the swab and seal the sample receiving chamber by inserting a closure device (e.g. a rubber bung) into the inlet (6). As described in more detail below, the disposable cartridge is then inserted into a processing unit, which can rotate the rotating central member (20).

By rotating the rotating central member (20), the lysis buffer (8) is directed into the interior annular chamber of the central member (20), and then directed into the sample receiving chamber (4). The lysis buffer is therefore brought into contact with the human or animal sample, and lyses cells therein to release DNA and RNA therefrom, and to provide a lysate. The lysis buffer and sample may be heated (e.g. by means of a processing unit into which the disposable cartridge is inserted) to facilitate DNA and/or RNA extraction. The lysate is then returned to the interior annular chamber of the central member (20), wherein it contacts the solid support to which DNA and/or RNA bind. Waste lysate may be discharged into a waste chamber. By further rotating the rotating central member (20), the wash buffer (10) is directed into the interior annular chamber of the central member (20) to wash to solid support, thereby removing cell debris from the DNA and/or RNA bound to the solid support. The used wash buffer is discharged from the interior annular chamber into a waste outlet (not illustrated). By further rotating the rotating central member (20), the elution buffer (10) is directed into the interior annular chamber of the central member (20) and contacts the solid support to elute DNA and/or RNA from the solid support and to provide a DNA/RNA eluent.

The DNA/RNA eluent comprising purified DNA and/or RNA is then brought into contact with lyophilised mixture (22), to provide a reconstituted mixture ready for RT-PCR and PCR amplification. The lyophilised mixture (22) comprises reverse transcriptase (RT), DNA polymerase, and dNTPs (dATP, dCTP, dGTP and dTTP). PCR dNTP mixtures and enzymes are commercially available from Empirical Bioscience, Inc. MI, USA. Those skilled in the art will be aware of suitable concentrations of reverse transcriptase, DNA polymerase and dNTPs required to provide suitable concentrations in the reconstituted mixture for RT-PCR and PCR in the reaction sites as described below.

The analysis unit comprises a plurality of reaction sites or wells within which the RT-PCR and/or PCR reaction occurs. The skilled person will appreciate that any number of reaction sites may be provided according to the nucleic acid sequences to be amplified and the number of replicates desired.

The reconstituted mixture comprising DNA and/or RNA, dNTPs, and enzymes for RT-PCR and PCR is directed out of the sample preparation unit of the disposable cartridge via outlet 24, and into the analysis unit section of the cartridge. A volume (e.g. 20-40 nL, e.g. around 30 nL) of the reconstituted mixture is dispensed into each of the reaction sites. The analysis unit is therefore in fluid communication with the sample preparation unit.

Each reaction site contains the desired primers and probes for RT-PCR (to generate cDNA) and DNA amplification. The primers and probes are provided at each reaction site in the form of a dried composition (e.g. dried by air-drying and/or lyophilisation). The skilled person being familiar with PCR will understand that probes are used to detect the presence of a specific DNA fragment through hybridization with at least one strand of double-stranded DNA; and that primers are used in the initiation of the polymerase chain reaction by hybridization with single-stranded DNA.

Each reaction site also contains a fluorescent marker such that as cDNA and/or DNA amplification progresses, the amplification can be evaluated in a qualitative and/or quantitative manner relative to a control. The fluorescence signal may be detected for instance, by means of a camera, and analysed using appropriate software. In embodiments, the primers and/or probes and/or dNTPs are labelled with fluorescent markers. In embodiments, fluorescent markers may include DNA-intercalating fluorescent dyes. Suitable fluorescent markers may include for example SYBR green™, 5′ 6-FAM (Fluorescein), or TaqMan™ (e.g TAMRA probe) assays, commercially available from ThermoFisher Scientific.

The plurality of reaction sites may also comprise one or more positive control reaction sites for confirming the RT-PCR or PCR reaction is progressing. In embodiments, amplification of the SNP gene of interest is taken as the positive control.

The analysis unit (AU) of the disposable cartridges, and the reaction sites thereof, according to the present invention are described in more detail below, by way of example only. As shown in FIGS. 1 and 2, the AU (4) may protrude from the housing of the cartridge, such that when the disposable cartridge is inserted into a processing unit (e.g. Nudgebox), the analysis unit can be monitored to evaluate progression of DNA amplification e.g. by means of a camera and evaluating fluorescence intensity from the reaction sites.

The AU comprises dried primers and probes uniquely spotted into 72 reaction wells (26) (also referred to as reaction sites) enabling multiplex analysis. The array in this example comprises six viral targets (IP2, IP4, e-gene, N1, N2, and N3); one SNP in the FTO gene, wherein the presence of the A allele indicates a higher risk or propensity to obesity and the presence of the T allele indicates a lower risk or propensity to obesity; and one wild-type SNP in the FTO gene. Each gene target has from three to nine technical replicates within the array, preferably from six to nine technical replicates. Following completion of the PCR reaction, the raw data (as determined by the processing unit e.g. Nudgebox) are input into a computer algorithm and processed [2]. The processed results are reported as positive, negative, indeterminate, invalid or aborted. According to the present example, the results of the test may be represented as shown in Table 1. In the Example according to Table 1, amplification of the FTO gene SNP is used as a positive control, but those skilled in the art will appreciate other positive controls may be used. Following the test, the single-use cartridge is disposed of following standard laboratory disposal procedures.

TABLE 1
Output according to the present example

| | |
|---|---|
| Invalid | Less than 2 of the 6 human FTO gene replicates have |
| | amplified. Viral results are not applicable |
| Positive for viral | At least 3 of the viral gene replicates and at least |
| infection + high | one replicate of the A allele of the FTO gene has |
| risk of a co- | amplified. The viral gene outputs could be 3 from the |
| morbidity (e.g. | same gene type, or 3 from different viral genes (e.g. |
| obesity) | N1, N2 and N3). At least 2 human FTO gene |
| | replicates have amplified. |
| Negative for viral | None of the viral gene replicates and at least one |
| infection + high | replicate of the A allele of the FTO gene have |
| risk of a co- | amplified. At least 2 human FTO gene replicates |
| morbidity (e.g. | have amplified. |
| obesity) | |
| Positive for viral | At least 3 of the viral gene replicates and at least |
| infection + low | one replicate of the T allele of the FTO gene have |
| risk of a co- | amplified. The viral gene outputs could be 3 from |
| morbidity (e.g. | the same gene type, or 3 from different viral genes |
| obesity) | (e.g. N1, N2 and N3). At least 2 human FTO gene |
| | replicates have amplified. |
| Negative for viral | None of the viral gene replicates and at least one |
| infection + low | replicate of the T allele of the FTO gene have |
| risk of a co- | amplified. At least 2 human FTO gene replicates have |
| morbidity (e.g. | amplified. |
| obesity) | |
| Indeterminate | One or two of the viral or FTP gene targets have |
| | amplified. At least 2 human FTO gene replicates |
| | have amplified. |
| Error | A technical issue (insufficient pressure) was detected |
| | during the test. The test was aborted. |

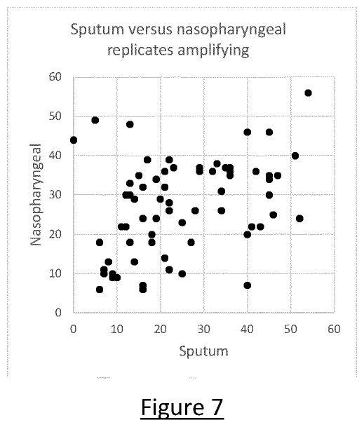

The single-use disposable cartridges according to the invention are configured to be used in conjunction with a processing unit (e.g. a NudgeBox) illustrated in FIG. 6. One or more disposable cartridges (also referred to herein as Nucleic Acid Cartridge) and the processing unit (e.g. a NudgeBox) together provide a DnaNudge platform. The cartridge is a disposable, sealed, and integrated lab-on-chip device that enables sample-to-result RT-PCR and PCR. As already described above, the cartridge consists of two main parts: an amplification unit (AU) and a sample preparation unit (SPU). A nasopharyngeal swab sample is inserted directly into the swab chamber of the sample preparation unit immediately after collection. The swab is broken, leaving the swab tip and the sample within the chamber, which is then sealed. Cartridges are placed in the (NudgeBox) processing unit, which provides the pneumatic, thermal, imaging, and mechanics required to run a real-time RT-PCR and PCR reaction outside of a laboratory setting.

Such a suitable processing unit (NudgeBox) is described in detail in WO2018055407. Once the disposable cartridge is inserted into the processing unit, the processing unit provides the necessary machinery to rotate the rotating central member (20) and thus provides the desired sequence of reagents/fluids, as described in detail above. More particularly, the disposable cartridge (e.g. Nucleic Acid Cartridge fits on top of a motor-driven spigot in the processing unit (e.g. NudgeBox™), which rotates the sample preparation unit (SPU) through each stage of RNA/DNA extraction processing. The reaction sites of the analysis unit (AU), inside which the RT-PCR and PCR reaction takes place, are then filled with the resulting solution comprising the RNA/DNA.

The processing unit may additionally provide heat to one or more chambers, and/or the central member (20) of the disposable cartridge to facilitate the DNA/RNA extraction process described above. Moreover, the processing unit may control the duration of each reaction step in the DNA/RNA extraction process. For example, the processing unit may control the temperature and duration for which the lysis buffer is in contact with the human or animal sample.

The processing unit may additionally provide heat to one or more reaction sites in the analysis unit of the disposable cartridge to facilitate RT-PCR/PCR. Moreover, the processing unit may control the duration of each reaction step in the RT-PCR and/or PCR process. For example, the processing unit may control the temperature and duration for the reverse-transcriptase step in RT-PCR, or the desired number of cycles for PCR. The RT step of RT-PCR is typically performed at 50° C. for 5 min. Denaturation is typically performed at 95° C. The temperature used in the denaturation step denatures the RT enzyme. PCR may be carried out for from 20 to 60 cycles, optionally from 30 to 50 cycles, for example 40 cycles.

The processing unit comprises a camera suitable for detecting fluorescence from the PCR reactions occurring in the analysis unit (AU) reaction sites. The processing unit can also comprise a computer program or algorithm configured to perform the RNA/DNA extraction process sequence and/or to perform RT-PCR/PCR. The computer program used to analyse the raw data is described below.

Analysis from individual wells is subdivided into model fitting, post-processing and classification stages. The data is modelled by the following formula:

$$f ( x ) = \frac{a}{1 + {e}^{- b ( x - c )}} + d + e x$$

where x is the PCR cycle. The first term, consisting of parameters a, b, and c (“the sigmoid term”) describe the exponential growth and decay in fluorescence intensity during a test. Parameters d and e account for system nonidealities, inter-test and inter-instrument variability. Raw data is fitted to the model with least-squares curve fitting techniques which provides estimates for parameters a, b and c. To ensure well-to-well and test-to-test consistency, data from each well undergoes drift correction and normalisation. Using the model parameters calculated previously the data is re-simulated with the e parameter set to zero and multiplying the remaining terms by a normalisation factor.

$$Norm [ f ( x ) ] = γ \cdot ( \frac{a}{1 + {e}^{- b ( x - c )}} + d + 0 \cdot x )$$

Where γ0 is the normalisation factor.

For a well to be classified as having amplified, the data should reflect the exponential growth and decay of a PCR reaction, simply, it should be “sigmoid-like”. This implies that the model parameters should fall within appropriate ranges. Specifically, inspection of the b and c parameters and the synthesis of two additional parameters (normalised sigmoid amplitude and r²) allow the algorithm to classify data as “sigmoid-like” or otherwise.

$$f ( x ) = \{ \begin{matrix}sigmoid , & \{ \begin{matrix}{b}_{1} < b < {b}_{2} \\ {c}_{1} < c < {c}_{2} \\ amp > {amp}_{th} \\ {r}^{2} > {r}_{th}^{2}\end{matrix} \\ sigmoid , & otherwise\end{matrix}$$

Where b₁, b₂, c₁, c₂ are upper and lower bounds for b and c respectively while ampₜₕ and rₜₕ² are thresholds over which the normalised amplitude and goodness of fit must exceed.

For a well to be classified as having amplified, the amplification curve should reflect the exponential growth and decay of a standard PCR reaction. A test is considered valid if at least two of six replicates of human FTO gene amplify, reflecting adequate sampling. If fewer than two (one or 0) replicates amplify, it is assumed that sample collection was inadequate, and the test is labelled as invalid. A positive test was defined as when at least three replicates of at least one viral gene target amplified; if 1 or 2 targets amplify the result is considered as indeterminate, otherwise a test was considered negative for SARS-CoV-2.

The FTO algorithm, implements similar calling logic to that of the COVID algorithm outlined above. However the FTO being SNPs, the logic for calling a test valid and the genotype call differs for this algorithm. FTO also is used as human control gene too. Specifically, there are 6 assays for each of the 2 FTO alleles A and T (as a convention, call the FTO assays B1 and B2). For a test to be valid, there needs to be at least 2 amplifying replicates of either B1 or B2 (or 2 of each). Provided this is the case, the FTO genotype is determined by counting the number of “pairwise c-differences”. A pairwise c-difference is the numerical distance between 2 “c” values of different pairs of B1 and B2. Given there are six replicates of B1 and B2, there are 36 unique pairs. If 2 “c” values are within a particular threshold (X) of one another, that pair is identified as a heterozygous pair (B1B2). However, if (for example) the B2 assay's “c” value is outside of X, the pair is labelled homozygous-B1 (B1B1). The opposite is true if B1 is outside of X, the pair is therefore homozygous-B2 (B2B2). If one assay of the pair has not amplified, its c value is assumed to be at infinity and therefore, by definition, the sample is classified as homozygous for the other assay. If both assays have not amplified, that pair is excluded from the count. Once all possible pairs have been assigned a label: B1B1, B1B2, B2B2, the pair with the highest count is assigned as the sample genotype.

### General Methods

### Obtaining Sputum Samples and Insertion Into Nucleic Acid Cartridge

### Equipment Required

SK-2 buccal swab with tube (Isohelix)
Oragene 500 sample collection tune (DNAgenotek)

i. The healthcare professional should prepare the patient for the procedure by asking them to sit upright, rinse their mouth with water and spit out prior to sputum collection.

ii. The patient should be asked to take a few deep breaths to help loosen secretions; please note, if patient is on a nebuliser, give nebuliser first and wait 10 minutes before taking a sample.

iii. The patient should cover their mouth before forcing out a deep cough to release the sputum. Sputum should be collected in the sample tube provided. Ideally the sputum sample should be no less than the size of a small fingernail.

iv. It is important that the healthcare professional checks the quality of the sputum to ensure it is not simply saliva, but rather sputum (mixture of phlegm and mucous). If the patient is unable to provide any sputum, advise to keep hydrated where possible, and encourage deep breathing to try again in an hour.

v. The sample tube should be held upright in one hand and the funnel lid closed with the other hand by pressing firmly until a loud click is heard. The liquid in the lid will be released into the tube to mix with the sputum.

vi. Holding the tube upright, unscrew the funnel from the tube and discard the funnel as clinical waste. Use the small screw cap to close the sample tube tightly. Shake the capped tube for 5 seconds.

vii. Remove the cap from the Isohelix swab while retaining the bung with stopper, and mix the swab in the sputum, rubbing gently for 10 seconds to get a good sputum sample on the swab. When extracting the swab from the sample tube, remove any excess sputum residue hanging from the swab by wiping the swab gently against the inside edge of the tube.

viii. Remove the cap from the DnaCartridge and insert the swab end at a vertical angle into the Cartridge (the Nucleic Acid Cartridge cap can be discarded) ix. Press the Isohelix cap with stopper into the Nucleic Acid Cartridge and gently remove tail of the swab, this will leave swab tip and sample in the swab chamber.

x. Discard the swab tail in a “sharps” bin.

xi. Lock the Nucleic Acid Cartridge closed with the Isohelix bung and run the test as per standard procedure.

### Reference Example 1-Test Accuracy for Viral RNA

The diagnostic accuracy of a disposable cartridge configured to test for SARS-CoV-2 alone (DnaNudge™ CovidNudge™) without SNP analysis, was assessed in April and May 2020 by comparing nasopharyngeal swab samples from individuals at three hospitals in London and Oxford against nasal and throat swabs tested on laboratory RT-PCR platforms [2]. The sensitivity of the DnaNudge point-of-care test compared with laboratory-based testing was 94% (95% CI 86-98) with a specificity of 100% (95% CI 99-100). Following this clinical validation, the SARS-CoV-2 test achieved the CE Mark in July 2020 enabling the technology to be used as standard of care in UK healthcare settings. Since July, over 20,000 patient samples have been tested on the CovidNudge platform across 8 separate hospital sites in London. Current NHS guidelines state that all patients admitted to UK hospitals must have a test for COVID-19; if a rapid test is not available, then patients presenting as emergency admissions must be isolated in side rooms until their laboratory test is returned, allowing the appropriate care pathway to be determined. These side rooms must then be fully cleaned regardless of the COVID-19 test result before they can be occupied by a subsequent patient, placing additional burden on an already stretched nursing resource. The deployment of CovidNudge as a point-of-care diagnostic has enabled effective triage and timely therapeutic and infection control interventions for emergency admission patients in clinical areas including adult and paediatric A&E, maternity, mental health and renal transplantation, and the technology has been fully embedded as an integral part of the emergency admission pathway at the deployment sites. With test results available within 90 minutes of sample collection, patients can be admitted into the appropriate care pathway bypassing the need for isolation if test results are negative, while enabling sites to meet operational targets to admit, transfer or discharge patients from A&E within 4 hours.

The disposable cartridge of the present invention has been optimised to allow simultaneous RT-PCR and PCR to detect one or more viral or bacterial nucleic acid sequences, and one or more SNPs associated with a disease or condition or risk of a disease or condition in a human or animal sample. The benefits provided by the present invention have been described above, and is exemplified in the examples provided below.

### Reference Example 2-Sputum Sampling and Test for Viral RNA

To investigate whether sputum samples are compatible with the DnaNudge platform, a validation study was undertaken comparing nasopharyngeal swab samples with sputum. Assessment took place using samples from two separate groups: patients admitted to hospital via the emergency department at Chelsea & Westminster NHS Foundation Trust, and members of the London Symphony Orchestra. Testing of emergency admissions at Chelsea & Westminster NHS Foundation Trust was done as a service evaluation approved by the point-of-care committee. Patients over 18 testing positive (n=71) and negative (n=103) on the DnaNudge platform via nasopharyngeal sampling agreed to provide a sputum sample. Members of the London Symphony Orchestra (n=118) were undergoing regular COVID-19 screening on the DnaNudge platform. All participants consented to supplying a sputum sample following the procedure outlined in the general methods above, in addition to providing a nasopharyngeal swab. Sputum samples were collected into a sample tube (Oragene500, DNAgenotek); the stabilising solution released by the Oragene collection tube has been shown to inactivate the SARS-CoV-2 virus due to the presence of an ionic detergent which renders ineffective enveloped viruses such as SARS-Cov-2 [3].

Following sputum and nasopharyngeal sample collection, the samples were tested on the DnaNudge platform. To test the sputum samples, an RNA/DNA buccal swab (SK-2, Isohelix) was used. The cap from the swab was removed while retaining the bung with stopper, and the swab was mixed in the sputum in stabilising solution by rubbing gently for 5 seconds to get a good sputum sample on the swab. When extracting the swab from the sample tube, any excess sputum residue hanging from the swab was removed by wiping the swab gently against the inside edge of the tube. The Isohelix swab was then inserted into the cartridge pressing the stopper in place, the swab tail was removed leaving the swab in the chamber, and the cartridge was sealed using the Isohelix bung (FIG. 2). The cartridge was then inserted into the NudgeBox and a test run following standard procedure [2]. 292 paired samples were obtained, and results are shown in Table 2.

TABLE 2
Nasopharyngeal and sputum paired samples tested on the
DnaNudge platform. Sputum samples demonstrated 98.6%
sensitivity (95% Cl = 92.4-99.96%) and 100% specificity
(95% Cl = 96.9-100%) against nasopharyngeal samples.

| | | SPUTUM SAMPLES | |
|---|---|---|---|
| | | POSITIVE | NEGATIVE |
| NASOPHARYNGEAL | POSITIVE | 70 | 1 |
| SAMPLES | NEGATIVE | 0 | 221 |

The relative concentration of viral load in nasal and sputum samples was quantitatively assessed by plotting the number of SARS-CoV-2 gene target replicates that amplified in each sample as shown in FIG. 7.

### Example 3-Disposable Cartridge for Detecting Viral RNA and SNPs

### Sample Preparation Unit

The disposable cartridge sample preparation unit comprised 450 μL lysis buffer, 450 μL wash buffer, and 1 mL elution buffer in separate chambers. The composition of the lysis buffer, wash buffer, and elution buffer were as follows:
Lysis Buffer: aqueous, pH 6; 66% 6M Guanidine hydrochloride (Sigma G3272; >99%); 25 mM sodium acetate (Sigma 71196; ˜3 M); 1 mM EDTA (Stock 10 mM); 0.1% Triton-X (1% Stock Solution).
Wash Buffer: aqueous, pH 7.5; 10 mM Trizma hydrochloride pH 7.5 (Sigma T2319, 1 L, 1M); 60% pure ethanol (Romil, A9314, ethanol absolute, 2.5 L).
Elution Buffer: aqueous, pH 8, 10 mM Trizma hydrochloride pH 8.0 (Sigma T3038, 1 L, 1M); 0.1 mM EDTA (10 mM Stock Solution); 0.1% Triton X (1% Stock Solution).

The sample preparation unit further comprised a PCR Master mix in the form of a lyophilised bead. PCR formulations for preparation of the Lyophilised beads were obtained from Empirical Biosciences, Inc. Lyophilised beads were obtained from Argonaut. The PCR formulations comprised the necessary dNTPs for PCR amplification, reverse transcriptase for RT-PCR, and DNA polymerase for PCR. The lyophilised beads further comprised Tris (pH 8.8), MgSO₄, KCl, dNTPs, bovine serum albumin (BSA), RNasin® Ribonuclease Inhibitor, Taq DNA polymerase, and reverse transcriptase.

Analysis Unit-Primers and Probes for Viral RNA Sequences and SNPs Primer mixes were prepared comprising the primers and probes indicated in the table below, to detect SARS-CoV-2. Primers and probes comprised in primer mixes N1-N3 were obtained from Centres for Disease Control & Prevention (CDC), USA. Primers and probes comprised in primer mix E were obtained from Charité-Universitätsmedizin, Berlin, Germany. Primers and probes comprised in primer mixes IP2 and IP4 were obtained from Institut Pasteur, Paris, France. All primers and probes are commercially available from Eurogentec, Belgium.

| | | | Primer/ |
|---|---|---|---|
| | Primer [F/R]/ | | Probe |
| Primer | Probe | | Concentra- |
| Mix | [P] Name | Sequence | tion (μM) |
| N1 | 2019-nCoV_N1-F | GAC CCC AAA ATC AGC GAA AT | 70 |
| | | (SEQ ID NO: 1) | |
| | 2019-nCoV_N1-R | TCT GGT TAC TGC CAG TTG AAT CTG | 70 |
| | | (SEQ ID NO: 2) | |
| | 2019-nCoV_N1-P | /56-FAM/ACC CCG CAT TAC GTT TGG | 17.5 |
| | | TGG ACC/36-TAMSp/(SEQ ID NO: 3) | |
| N2 | 2019-nCoV_N2-F | TTA CAA ACA TTG GCC GCA AA | 70 |
| | | (SEQ ID NO: 4) | |
| | 2019-nCoV_N2-R | GCG CGA CAT TCC GAA GAA | 70 |
| | | (SEQ ID NO: 5) | |
| | 2019-nCoV_N2-P | /56-FAM/ACA ATT TGC CCC CAG CGC | 17.5 |
| | | TTC AG/36-TAMSp/(SEQ ID NO: 6) | |
| N3 | 2019-nCoV_N3-F | GGG AGC CTT GAA TAC ACC AAA A | 70 |
| | | (SEQ ID NO: 7) | |
| | 2019-nCoV_N3-R | TGT AGC ACG ATT GCA GCA TTG | 70 |
| | | (SEQ ID NO: 8) | |
| | 2019-nCoV_N3-P | /56-FAM/AYC ACA TTG GCA CCC GCA | 27.5 |
| | | ATC CTG/36-TAMSp/(SEQ ID NO: 9) | |
| E | E_Sarbeco_F1 | ACAGGTACGTTAATAGTTAATAGCGT | 14 |
| | | (SEQ ID NO: 10) | |
| | E_Sarbeco_R2 | ATATTGCAGCAGTACGCACACA | 14 |
| | | (SEQ ID NO: 11) | |
| | E_Sarbeco_P1 | /56- | 7 |
| | | FAM/ACACTAGCCATCCTTACTGCGCTTCG/ | |
| | | 36-TAMSp/(SEQ ID NO: 12) | |
| IP2 | nCoV_IP2-12669Fw | ATGAGCTTAGTCCTGTTG (SEQ ID | 31.5 |
| | | NO: 13) | |
| | nCoV_IP2-12759Rv | CTCCCTTTGTTGTGTTGT (SEQ ID | 31.5 |
| | | NO: 14) | |
| | nCoV_IP2- | /56-FAM/AGATGTCTTGTGCTGCCGGTA/36- | 10.5 |
| | 12696bProbe(+) | TAMSp/(SEQ ID NO: 15) | |
| IP4 | nCoV_IP4-14059FW | GGTAACTGGTATGATTTCG (SEQ ID | 31.5 |
| | | NO: 16) | |
| | nCoV_IP4-14146Rv | CTGGTCAAGGTTAATATAGG (SEQ ID | 31.5 |
| | | NO: 17) | |
| | nCoV_IP4- | /56-FAM/TCATACAAACCACGCCAGG/36- | 10.5 |
| | 14084Probe(+) | TAMSp/(SEQ ID NO: 18) | |

For SNPs, primer mixes were prepared comprising the primers and probes indicated in the table below to detect SNPs in the FTO gene.

| | | | Primer/ |
|---|---|---|---|
| | Primer | | Probe |
| | [F/R]/ | | Concen- |
| Primer | Probe [P] | | tration |
| Mix | Name | Sequence | (μM) |
| NUT01.13 | SNP01.01- | CAGGGCCATTTATCTATGAGACACTACAG | 15.75 |
| (T allele) | FWD | (SEQ ID NO: 19) | |
| | SNP01.02- | GTTGCAGCAATAACCTACCTTAA (SEQ ID | 15.75 |
| | REV-T | NO: 20) | |
| | (rs1558902) | | |
| | SNP01.12L | /56- | 5.25 |
| | probe | FAM/TGTA[+A]ACCC[+A]CAGG[+G]CTAG[+A]CACA[+A]TG | |
| | | C/36-TAMSp/(SEQ ID NO: 21) | |
| NUT01.14 | SNP01.01- | CAGGGCCATTTATCTATGAGACACTACAG | 15.75 |
| (A allele) | FWD | (SEQ ID NO: 19) | |
| | SNP01.03- | GTTGCAGCAATAACCTACCTTAT (SEQ ID NO: 22) | 15.75 |
| | REV-A | | |
| | (rs1558902) | | |
| | SNP01.12L | /56- | 5.25 |
| | probe | FAM/TGTA[+A]ACCC[+A]CAGG[+G]CTAG[+A]CACA[+A]TG | |
| | | C/36-TAMSp/(SEQ ID NO: 21) | |

### Analysis Unit-Reaction Site Pattern for Viral RNA and SNP Detection

Assays were arranged in the analysis unit in a 72-well plate type arrangement (see FIG. 5) to provide a plurality of reaction sites, by adding the desired primer mix to each reaction site. The following table provides information regarding the assays used in the analysis unit.

| Assay No. | Assay (Primer Mix) | Reaction Site Replicates |
|---|---|---|
| 1 | COV.N1 (N1) | 9 |
| 2 | COV.N2 (N2) | 9 |
| 3 | COV.N3 (N3) | 9 |
| 4 | COV.IP2 (IP2) | 9 |
| 5 | COV.IP4 (IP4) | 9 |
| 6 | COV.E (E) | 9 |
| 7 | FTOwt_NUT01.13_T | 6 |
| | (NUT01.13) | |
| 8 | FTOmut_NUT01.14_A | 6 |
| | (NUT01.14) | |
| blank | | 6 |

Primer mixes were prepared as aqueous solutions comprising 0.3% vol/vol Tween-20, and 30 nL of the desired primer mix was added to each reaction well. The primer mix aqueous solution in each reaction well was air dried therein for 30 minutes at 37° C. Scienion (Germany) precision dispensing apparatus was used for dispensing and drying the primer and probe mixtures in the reaction wells.

Blank wells comprised no primer mix. The total number of reaction sites was 72.

As described above, the mixture comprising the lyophilised bead composition and nucleic acid samples is prepared in the sample preparation unit, and said mixture is then dispensed into each reaction well. A typical composition of said mixture comprising the lyophilised bead composition and nucleic acid samples is provided below, and the final concentration in each reaction site is indicated.

| | Final concentration per reaction site | |
|---|---|---|
| Composition of Sample | Conc. | Units |
| Tris pH 8.8 | 10 | mM |
| MgSO4 | 7.5 | mM |
| KCl | 50 | mM |
| dNTPs | 0.1 | mM |
| BSA | 0.5 | mg/ml |
| RNasin ® Ribonuclease Inhibitor | 0.25 | U/ul |
| Taq DNA Polymerase | 0.25 | U/ul |
| Reverse Transcriptase | 1.25 | U/ul |
| Nucleic acid sample | 200 | copies/ul |
| | 1 | X |

### Example 4-Conditions for RT-PCR and PCR

### RT-PCR and PCR

Optimised PCR conditions for both viral nucleic acid and human DNA detection (RT-PCR and PCR) were determined to be as follows:

| | | | Temperature | | |
|---|---|---|---|---|---|
| Step | | #Cycles | (° C.) | Time | |
| Reverse transcriptase (RT) | | 1 | 50 | 5 | minutes |
| RT inactivation and activation | | 1 | 95 | 20 | seconds |
| of DNA polymerase | | | | | |
| PCR | Denaturation | 40 | 95 | 2 | seconds |
| | Annealing/Extension | | 60 | 30 | seconds |

The disposable cartridge and NudgeBox™ processing unit were therefore configured to subject the analysis unit to the indicated temperatures for the indicated times, and for the indicated number of cycles.

### Example 5-Sars-CoV-2 Analysis and FTO Analysis Saliva-swab samples were gathered from 50 different human individuals and a selected sample (10 samples) were spiked with Sars-Cov-2 by dipping each selected swab into a sample comprising Sars-Cov-2. Analysis of the swabs was performed using a disposable cartridge according to Example 3, having a reaction site pattern as described in Example 3 and as depicted in FIG. 5. The disposable cartridge was inserted into a NudgeBox™ processing unit for analysis.

Eyeball analysis of the raw data was performed by the user, in which the user inspected the raw data to determine whether amplification has occurred. As described previously, successful DNA amplification is expected to show an exponential increase in the number of amplicons. This process was repeated to train and optimise algorithm described above. The calling results of the optimised algorithm and eyeball match analysis is outlined in the table below. FTO SNP of the human sample was analysed by standard Sanger sequencing methods.

Results (viral positive (+ve) or viral negative (−ve); obesity genotype) were assessed according to the criteria set out in Table 1 above. Obesity genotypes TA and AA were categorised as a high-risk obesity phenotype. Obesity genotypes TT were categorised as a low-risk obesity phenotype.

| | | | | | | | | | | | | | | algorithm |
|---|---|---|---|---|---|---|---|---|---|---|---|---|---|---|
| | Number of Replicates | | | | | | | | | Cartridge | Cartridge | | Eye | vs |
| Sample | FTO | | | Viral (Sars-Cov-2) | | | | | | Viral | Genotype | Truth | balling | eyeball |
| No. | NUT01_13 | NUT01_14 | blank | e-gene | ip2 | ip4 | n1 | n2 | n3 | Call | Call | Genotype | Genotype | match |
| 1 | 4 | 3 | 0 | 0 | 0 | 0 | 0 | 0 | 2 | −ve | TA | TA | TA | TRUE |
| 2 | 4 | 4 | 0 | 0 | 0 | 0 | 0 | 0 | 0 | −ve | TA | TA | TA | TRUE |
| 3 | 4 | 4 | 0 | 0 | 0 | 0 | 0 | 0 | 0 | −ve | TA | TA | TA | TRUE |
| 4 | 6 | 2 | 0 | 0 | 0 | 0 | 0 | 0 | 1 | −ve | TT | TT | TT | TRUE |
| 5 | 6 | 4 | 0 | 0 | 0 | 0 | 0 | 0 | 1 | −ve | TT | TT | TT | TRUE |
| 6 | 5 | 3 | 0 | 0 | 0 | 0 | 0 | 0 | 1 | −ve | TT | TT | TT | TRUE |
| 7 | 5 | 5 | 0 | 0 | 0 | 0 | 0 | 1 | 2 | +ve | TA | TA | TA | TRUE |
| 8 | 6 | 4 | 0 | 0 | 0 | 0 | 0 | 0 | 3 | +ve | TT | TT | TT | TRUE |
| 9 | 4 | 1 | 0 | 0 | 0 | 0 | 0 | 0 | 0 | −ve | TT | TT | TT | TRUE |
| 10 | 5 | 0 | 0 | 0 | 0 | 0 | 0 | 0 | 1 | −ve | TT | TT | TT | TRUE |
| 11 | 6 | 1 | 0 | 0 | 0 | 0 | 0 | 0 | 1 | −ve | TT | TT | TT | TRUE |
| 12 | 0 | 5 | 0 | 0 | 0 | 0 | 0 | 0 | 1 | −ve | AA | AA | AA | TRUE |
| 13 | 1 | 1 | 0 | 0 | 0 | 0 | 0 | 0 | 1 | −ve | TA | TA | TA | TRUE |
| 14 | 1 | 1 | 0 | 0 | 0 | 0 | 0 | 0 | 2 | −ve | TT | TT | TT | TRUE |
| 15 | 2 | 0 | 0 | 0 | 0 | 0 | 0 | 0 | 1 | −ve | TT | TT | TT | TRUE |
| 16 | 5 | 5 | 0 | 0 | 0 | 0 | 0 | 0 | 2 | −ve | TA | TA | TA | TRUE |
| 17 | 4 | 2 | 0 | 0 | 0 | 0 | 0 | 0 | 1 | −ve | TT | TT | TT | TRUE |
| 18 | 3 | 3 | 0 | 0 | 0 | 0 | 0 | 0 | 0 | −ve | TA | TA | TA | TRUE |
| 19 | 0 | 5 | 0 | 0 | 0 | 0 | 0 | 0 | 0 | −ve | AA | AA | AA | TRUE |
| 20 | 1 | 1 | 0 | 0 | 0 | 1 | 0 | 0 | 1 | −ve | TA | TT | TA | FALSE |
| 21 | 4 | 4 | 0 | 0 | 0 | 0 | 0 | 0 | 0 | −ve | TA | TA | TA | TRUE |
| 22 | 6 | 0 | 0 | 0 | 0 | 0 | 0 | 0 | 0 | −ve | TT | TT | TT | TRUE |
| 23 | 2 | 2 | 0 | 0 | 0 | 0 | 0 | 0 | 0 | −ve | TA | TA | TA | TRUE |
| 24 | 3 | 0 | 0 | 0 | 0 | 0 | 0 | 0 | 1 | −ve | TT | TT | TT | TRUE |
| 25 | 0 | 1 | 0 | 0 | 0 | 0 | 0 | 0 | 0 | −ve | AA | TT | AA | FALSE |
| 26 | 0 | 1 | 0 | 0 | 0 | 0 | 0 | 0 | 0 | −ve | AA | TA | TA | TRUE |
| 27 | 3 | 2 | 0 | 0 | 0 | 0 | 0 | 0 | 2 | −ve | TT | TT | TT | TRUE |
| 28 | 0 | 4 | 0 | 0 | 0 | 0 | 0 | 0 | 0 | −ve | AA | AA | AA | TRUE |
| 29 | 6 | 2 | 0 | 0 | 0 | 0 | 0 | 0 | 3 | +ve | TT | TT | TT | TRUE |
| 30 | 5 | 3 | 0 | 0 | 0 | 0 | 0 | 0 | 3 | +ve | TT | TT | TT | TRUE |
| 31 | 5 | 0 | 0 | 0 | 0 | 0 | 0 | 0 | 1 | −ve | TT | TT | TT | TRUE |
| 32 | 0 | 1 | 0 | 0 | 0 | 0 | 0 | 0 | 1 | −ve | AA | TA | AA | FALSE |
| 33 | 5 | 1 | 0 | 0 | 0 | 0 | 0 | 0 | 1 | −ve | TT | TT | TT | TRUE |
| 34 | 1 | 2 | 0 | 0 | 0 | 0 | 0 | 0 | 3 | +ve | AA | TT | TT | TRUE |
| 35 | 5 | 0 | 0 | 0 | 0 | 0 | 0 | 0 | 0 | −ve | TT | TT | TT | TRUE |
| 36 | 3 | 1 | 0 | 0 | 0 | 0 | 0 | 0 | 1 | −ve | TT | TT | TT | TRUE |
| 37 | 4 | 4 | 0 | 4 | 1 | 1 | 5 | 6 | 6 | +ve | TA | TA | TA | TRUE |
| 38 | 4 | 0 | 0 | 2 | 3 | 1 | 6 | 3 | 4 | +ve | TT | TT | TT | TRUE |
| 39 | 5 | 0 | 0 | 0 | 1 | 1 | 1 | 3 | 1 | +ve | TT | TT | TT | TRUE |
| 40 | 1 | 6 | 0 | 2 | 0 | 0 | 3 | 8 | 5 | +ve | AA | AA | AA | TRUE |
| 41 | 5 | 0 | 0 | 5 | 4 | 1 | 10 | 8 | 8 | +ve | TT | TT | TT | TRUE |
| 42 | 5 | 4 | 0 | 3 | 2 | 1 | 9 | 6 | 4 | +ve | TA | TA | TA | TRUE |
| 43 | 1 | 3 | 0 | 3 | 3 | 1 | 9 | 8 | 6 | +ve | AA | TA | TA | TRUE |
| 44 | 6 | 3 | 0 | 4 | 4 | 3 | 8 | 8 | 9 | +ve | TT | TT | TT | TRUE |
| 45 | 5 | 4 | 0 | 3 | 1 | 3 | 10 | 7 | 8 | +ve | TT | TT | TT | TRUE |
| 46 | 5 | 5 | 0 | 3 | 3 | 2 | 7 | 9 | 8 | +ve | TA | TA | TA | TRUE |
| 47 | 2 | 2 | 0 | 0 | 0 | 0 | 0 | 0 | 1 | −ve | TA | TA | TA | TRUE |
| 48 | 6 | 2 | 0 | 0 | 0 | 0 | 0 | 0 | 2 | −ve | TT | TT | TT | TRUE |
| 49 | 2 | 2 | 0 | 0 | 0 | 0 | 0 | 0 | 0 | −ve | TA | TA | TA | TRUE |
| 50 | 0 | 3 | 0 | 0 | 0 | 0 | 0 | 0 | 0 | −ve | AA | AA | AA | TRUE |

Control experiments for Examples 26, 27 and 33 were invalid, which accounts for 2 of the 3 experiments where the algorithm call was incorrect. Control experiments for the remaining experiments were valid. A summary of the data above is provided in the table below, which details the number of samples tested, and the overall genotype calling accuracy (represented as %) of the disposable cartridges used for each sample is indicated.

| | Total | Correct | Correct |
|---|---|---|---|
| | number | call | Call (%) |
| Human sample (Covid | 50 | 47 | 96 |
| positive + Covid negative) | | | |
| Human sample (Covid positive) | 10 | 10 | 100 |

### References

[1] Lisboa Bastos M. et al, Diagnostic accuracy of serological tests for covid-19: systematic review and meta-analysis. BMJ 2020;370: m2516 | doi: 10.1136/bmj.m2516
[2] Gilbani M., Toumazou C. et al. Assessing a novel, lab-free, point-of-care test for SARS-CoV-2 (CovidNudge): a diagnostic accuracy study. Lancet Microbe. https://doi.org/10.1016/S2666-5247(20)30121-X
[3] https://www.dnagenotek.com/US/pdf/MK-01430.pdf

1. A disposable cartridge comprising:
a sample receiving chamber for receiving a human or animal sample;
one or more further chambers containing reagents and fluids; and
an analysis unit (AU) having a multiplicity of reaction sites;
the cartridge being operable to move fluids between the chambers and the AU and thereby generate at said reaction sites a detectable indication of the presence in said sample of (a) one or more viral or bacterial nucleic acid sequences; and (b) one or more defined single nucleotide polymorphisms in the human or animal genome; and
wherein the one or more SNPs are indicative of a disease or a disease risk or other physiological condition in the human or animal, and wherein the disease or physiological condition is indicative of a poorer clinical outcome associated with a disease or other condition caused in the human or animal by the virus or bacteria.

2. A disposable cartridge according to claim 1, wherein the cartridge is a single-use disposable cartridge.

3. A disposable cartridge according to claim 1, wherein the cartridge is configured to perform single nucleotide polymorphism genotyping; and to detect one or more viral or bacterial nucleic acid sequences.

4. A disposable cartridge according to claim 3, wherein the cartridge is configured to detect one or more viral nucleic acid sequences, wherein the viral nucleic acid is RNA.

5. A disposable cartridge according to claim 4, wherein the cartridge is configured to generate complementary DNA (cDNA) from the one or more viral RNA sequences.

6. A disposable cartridge according to claim 5 wherein the cartridge is configured to generate complementary DNA (cDNA) from one or more viral RNA sequences; and then to perform DNA amplification of said cDNA concurrently with DNA amplification of said one or more defined single nucleotide polymorphisms in the DNA of the human or animal sample.

7. A disposable cartridge according to claim 1 wherein the disease or other condition caused by the virus or bacteria infection is a respiratory disease.

8. A disposable cartridge according to claim 7 wherein the respiratory disease is a viral infection and the virus is a common cold, influenza, respiratory syncytial virus, adenovirus, or coronavirus.

9. A disposable cartridge according to claim 8 wherein the virus is a coronavirus, optionally SARS, SARS-CoV-2 or MERS, optionally still wherein the virus is SARS-CoV-2.

10. A disposable cartridge according to claim 1:
wherein at least one reaction site comprises at least one nucleic acid probe sequence capable of detecting at least one viral or bacterial nucleic acid sequence; and
wherein at least one reaction site comprises at least one SNP nucleic acid probe sequence, capable of detecting the one or more SNPs in the human or animal genome.

11. A disposable cartridge of claim 10, wherein the at least one reaction sites comprise at least one or more primer nucleic acid sequences.

12. A disposable cartridge according to claim 10, wherein the least one nucleic acid probe sequence capable of detecting at least one viral or bacterial nucleic acid is a probe sequence capable of detecting at least one viral nucleic acid, optionally wherein the at least one viral nucleic acid comprises at least a portion of a nucleic acid sequence of SARS-CoV-2.

13. A disposable cartridge according to claim 10, wherein the at least one SNP nucleic acid probe sequence is capable of detecting one or more SNPs associated with fat mass and obesity-associated (FTO) gene sequences, or specific fragments thereof.

14. A disposable cartridge according to claim 13 wherein the at least one SNP nucleic acid probe sequence is capable of binding to at least one nucleic acid sequence selected from rs9937053 (A/G), rs9939973 (A/G), rs9940128 (A/G), rs1421085 (C/T), rs1558902 (A/T), rs1121980 (A/G), rs7193144 (C/T), rs8043757 (T/A), rs8050136 (A/C), rs3751812 (T/G), rs9923233 (C/G), rs9926289 (A/G), rs9939609 (A/T), rs7185735 (G/A), rs9931494 (G/C), rs17817964 (T/C), rs9930506 (G/A), rs9932754 (C/T), rs9922619 (T/G), rs7204606 (C/T) and rs12149832 (A/G) alleles, optionally wherein the least one nucleic acid sequence is rs1558902 (A/T).

15. A disposable cartridge according to claim 1, wherein the one or more single nucleotide polymorphisms (SNPs) in the human or animal genome to be detected comprises at least 2, 3, 4, 5, 6, 7, or 8 specific nucleic acid sequences which are specific to the disease or other physiological condition in the human or animal, optionally wherein the one or more SNPs to be detected comprises at most 4, 6, 8, 10, or 12 specific nucleic acid sequences which are specific to the disease or other physiological condition in the human or animal.

16. A disposable cartridge according to claim 1 wherein the disease or other physiological condition in the human or animal is one or more of obesity, diabetes, cardiovascular disease, cerebrovascular disease, respiratory disease, kidney disease and malignancy.

17. A disposable cartridge according to claim 16, wherein the disease or other physiological condition in the human or animal is obesity, and wherein the one or more SNPs is, or includes, fat mass and obesity-associated (FTO) gene sequences, or specific fragments thereof.

18. A disposable cartridge according to claim 1, wherein the sample is nasopharyngeal or sputum or saliva.

19. A disposable cartridge according to claim 1, wherein the cartridge is configured to be introduced into a processing unit, optionally wherein the processing unit is a NudgeBox™ analyser.

20. A disposable cartridge according to claim 1 wherein the one or more further chambers comprise at least a chamber comprising a lysis buffer, a chamber containing a wash buffer, a chamber containing an elution buffer, and a chamber comprising a lyophilised composition comprising reagents for RT-PCR and PCR.

21. A disposable cartridge according to claim 20 wherein the lysis buffer comprises:
at least one chaotropic agent, wherein the concentration of the chaotropic agent in the lysis buffer is from 2 M to 6 M;
at least one acetate salt, wherein the concentration of the acetate salt in the lysis buffer is from 0.1 to 2 mM;
at least one chelating agent, wherein the concentration of the chelating agent in the lysis buffer is from 5 mM to 50 mM; and
at least one surfactant, wherein the concentration of the surfactant in the lysis buffer is from 0.1 to 2 mM.

22. A disposable cartridge according to claim 21:
(i) wherein the lysis buffer is an aqueous lysis buffer, and wherein the lysis buffer has a pH of from 5.0-7.0, preferably 5.5-6.5, and preferably still about 6.0; and/or
(ii) wherein the chaotropic agent is guanidine hydrochloride; and/or
(iii) wherein the acetate salt is sodium acetate; and/or
(iv) wherein the chelating agent is EDTA.

23. A disposable cartridge according to claim 20, wherein the wash buffer comprises from 0.1 to 20 mM tris(hydroxymethyl)aminomethane and from 50 to 90 wt % of a C₁-C₃ alcohol, preferably from 60 to 80 wt % of a C₁-C₃ alcohol.

24. A disposable cartridge according to claim 23:
(i) wherein the C₁-C₃ alcohol is ethanol; and/or
(ii) wherein the wash buffer is an aqueous wash buffer, and wherein the wash buffer has a pH of from 6.0-9.0, preferably 7.0-8.0, and preferably still about 7.5.

25. A disposable cartridge according to claim 20:
(i) wherein the elution buffer comprises from 0.1 to 20 mM tris(hydroxymethyl)aminomethane; at least one chelating agent, wherein the concentration of the chelating agent in the lysis buffer is from 0.01 mM to 1 mM: and at least one surfactant, wherein the concentration of the surfactant in the lysis buffer is from 0.1 to 2 mM, optionally wherein the chelating agent is EDTA; and/or
(ii) wherein the elution buffer is an aqueous elution buffer, and wherein the elution buffer has a pH of from 6.0 to 10.0, preferably from 7.0 to 9.0, and preferably still about 8.0.

26. A method for detecting the presence of (a) one or more viral or bacterial nucleic acid sequences: and (b) one or more defined single nucleotide polymorphisms (SNPs) in the human or animal genome, in a human or animal sample, the method comprising:
obtaining the human or animal sample;
inserting the sample into the sample receiving chamber of a disposable cartridge according to claim 1, and sealing the sample within the sample receiving chamber;
inserting the cartridge into a processing unit, optionally wherein the processing unit is a NudgeBox™ analyser;
extracting RNA and DNA from the human or animal sample; and
performing RT-PCR and PCR reactions on the sample within the disposable cartridge;
wherein the one or more SNPs are indicative of a disease or a disease risk or other physiological condition in the human or animal, and wherein the disease or physiological condition is indicative of a poorer clinical outcome associated with a disease or other condition caused in the human or animal by the virus or bacteria; and
wherein the processing unit is programmed to concurrently perform RT-PCR and PCR reactions on the sample within the disposable cartridge; and to analyse the detectable indication.

27. A system for detecting the presence of (a) one or more viral or bacterial nucleic acid sequences; and (b) one or more defined single nucleotide polymorphisms (SNPs) in the human or animal genome, in a human or animal sample, the system comprising:
a disposable cartridge according to claim 1; and
a processing unit, wherein the processing unit is configured to effect extraction of nucleic acids from the human or animal sample inside the disposable cartridge, effect RT-PCR and PCR on said nucleic acids inside the disposable cartridge, and to detect formation of amplicons in arising from PCR.


SEQUENCE LISTING (24 sequences)

SEQ ID NO: 1 - RNA, 20 nt, synthetic construct
  source 1..20: mol_type = other RNA
gaccccaaaatcagcgaaat

SEQ ID NO: 2 - RNA, 24 nt, synthetic construct
  source 1..24: mol_type = other RNA
tctggttactgccagttgaatctg

SEQ ID NO: 3 - RNA, 24 nt, synthetic construct
  source 1..24: mol_type = other RNA
accccgcattacgtttggtggacc

SEQ ID NO: 4 - RNA, 20 nt, synthetic construct
  source 1..20: mol_type = other RNA
ttacaaacattggccgcaaa

SEQ ID NO: 5 - RNA, 18 nt, synthetic construct
  source 1..18: mol_type = other RNA
gcgcgacattccgaagaa

SEQ ID NO: 6 - RNA, 23 nt, synthetic construct
  source 1..23: mol_type = other RNA
acaatttgcccccagcgcttcag

SEQ ID NO: 7 - RNA, 22 nt, synthetic construct
  source 1..22: mol_type = other RNA
gggagccttgaatacaccaaaa

SEQ ID NO: 8 - RNA, 21 nt, synthetic construct
  source 1..21: mol_type = other RNA
tgtagcacgattgcagcattg

SEQ ID NO: 9 - RNA, 24 nt, synthetic construct
  source 1..24: mol_type = other RNA
aycacattggcacccgcaatcctg

SEQ ID NO: 10 - RNA, 26 nt, synthetic construct
  source 1..26: mol_type = other RNA
acaggtacgttaatagttaatagcgt

SEQ ID NO: 11 - RNA, 22 nt, synthetic construct
  source 1..22: mol_type = other RNA
atattgcagcagtacgcacaca

SEQ ID NO: 12 - RNA, 26 nt, synthetic construct
  source 1..26: mol_type = other RNA
acactagccatccttactgcgcttcg

SEQ ID NO: 13 - RNA, 18 nt, synthetic construct
  source 1..18: mol_type = other RNA
atgagcttagtcctgttg

SEQ ID NO: 14 - RNA, 18 nt, synthetic construct
  source 1..18: mol_type = other RNA
ctccctttgttgtgttgt

SEQ ID NO: 15 - RNA, 21 nt, synthetic construct
  source 1..21: mol_type = other RNA
agatgtcttgtgctgccggta

SEQ ID NO: 16 - RNA, 19 nt, synthetic construct
  source 1..19: mol_type = other RNA
ggtaactggtatgatttcg

SEQ ID NO: 17 - RNA, 20 nt, synthetic construct
  source 1..20: mol_type = other RNA
ctggtcaaggttaatatagg

SEQ ID NO: 18 - RNA, 19 nt, synthetic construct
  source 1..19: mol_type = other RNA
tcatacaaaccacgccagg

SEQ ID NO: 19 - RNA, 29 nt, synthetic construct
  source 1..29: mol_type = other RNA
cagggccatttatctatgagacactacag

SEQ ID NO: 20 - RNA, 23 nt, synthetic construct
  source 1..23: mol_type = other RNA
gttgcagcaataacctaccttaa

SEQ ID NO: 21 - RNA, 28 nt, synthetic construct
  source 1..28: mol_type = other RNA
  misc_difference 5: note = optional insertion
  misc_difference 10: note = optional insertion
  misc_difference 15: note = optional insertion
  misc_difference 20: note = optional insertion
  misc_difference 25: note = optional insertion
tgtaaacccacagggctagacacaatgc

SEQ ID NO: 22 - RNA, 29 nt, synthetic construct
  source 1..29: mol_type = other RNA
cagggccatttatctatgagacactacag

SEQ ID NO: 23 - RNA, 23 nt, synthetic construct
  source 1..23: mol_type = other RNA
gttgcagcaataacctaccttat

SEQ ID NO: 24 - RNA, 28 nt, synthetic construct
  source 1..28: mol_type = other RNA
  misc_difference 5: note = optional insertion
  misc_difference 10: note = optional insertion
  misc_difference 15: note = optional insertion
  misc_difference 20: note = optional insertion
  misc_difference 25: note = optional insertion
tgtaaacccacagggctagacacaatgc